(19) United States
(12) Patent Application Publication
(10) Pub. No.: US 2026/0011449 A1
(43) Pub. Date: Jan. 8, 2026

(54) TRAINING DEVICE, STATE PREDICTION DEVICE, TRAINING METHOD, AND PROGRAM

(71) Applicant: NEC Corporation, Tokyo (JP)

(72) Inventors: Yuki KOSAKA, Tokyo (JP); Mana Hashimoto, Tokyo (JP)

(73) Assignee: NEC Corporation, Tokyo (JP)

(21) Appl. No.: 19/244,092

(22) Filed: Jun. 20, 2025

(30) Foreign Application Priority Data
Jul. 3, 2024 (JP) 2024-107384

(51) Int. Cl. G16H 50/30; G16H 20/00; G16H 50/70

(52) U.S. Cl. CPC G16H 50/30; G16H 20/00; G16H 50/70

(57) ABSTRACT

The training device 1X mainly includes a type determination means 16X and a training means 17X. The type determination means 16X determines a type of each of subjects related to an action based on a recommended action, a type of the action, or the combination thereof, and an actual action, a type of the action, or the combination thereof. The training means 17X trains a state prediction model, for each type related to the action, based on a lifelog of each of the subjects classified by the type, wherein the state prediction model is obtained by performing machine learning of a relationship between a lifelog and a predicted index value of a state to be predicted of a person for which the lifelog has been measured. The prediction result output by the state prediction model is used for decision making, for example

### INCORPORATION BY REFERENCE

This application is based upon and claims the benefit of priority from Japanese Patent Application No. 2024-107384, filed on Jul. 3, 2024, the disclosure of which is incorporated herein in its entirety by reference.

### TECHNICAL FIELD

The present disclosure relates to a technical field of a training device, a state prediction device, a training method, and a program related to prediction of a state of a subject.

### BACKGROUND

Devices or systems for predicting a state of a subject for the purpose of health promotion or the like have been known. For example, WO 2019/116679 A1 discloses a system that predicts a figure and a health condition after a lapse of a predetermined period of time based on information regarding a lifestyle, a dietary habit, and the like of a user with reference to current figure information and current health conditions of the user.

### SUMMARY

When prediction accuracy in predicting the state of the user is low, the user who uses the system feels that the state does not change as predicted, whereby the user may be less motivated to continuously use the system.

In view of the problem described above, an object of the present disclosure is to provide a training device, a state prediction device, a training method, and a program related to training of a model for highly accurately predicting a state of a subject or state prediction using the model.

In an example aspect of the present disclosure, there is provided a training device including:
a type determination means for determining a type of each of subjects related to an action based on a recommended action, which is an amount of the action recommended to each of the subjects, a type of the action, or a combination of the amount of the action and the type of the action, and an actual action, which is an actual amount of the action of each of the subjects, a type of the action, or a combination of the actual amount of the action and the type of the action; and
a training means for training a state prediction model, for each type related to the action, based on a lifelog of each of the subjects classified by the type, wherein
the state prediction model is obtained by performing machine learning of a relationship between a lifelog and a predicted index value of a state to be predicted of a person for which the lifelog has been measured.

In an example aspect of the present disclosure, there is provided a state prediction device including:
a type determination means for determining a type of a subject related to an action based on a recommended action, which is an amount of the action recommended to the subject, a type of the action, or a combination of the amount of the action and the type of the action, and an actual action, which is an actual amount of the action of the subject, a type of the action, or a combination of the actual amount of the action and the type of the action;
a model selection means for selecting a state prediction model to be used in state prediction of the subject from state prediction models trained for each type related to the action based on the determined type; and
a state prediction means for predicting a state of the subject based on the selected state prediction model and a lifelog of the subject, wherein
the state prediction model is obtained by performing machine learning of a relationship between a lifelog and a predicted index value of a state to be predicted of a person for which the lifelog has been measured.

In an example aspect of the present disclosure, there is provided a training method executed by a computer, including:
determining a type of each of subjects related to an action based on a recommended action, which is an amount of the action recommended to each of the subjects, a type of the action, or a combination of the amount of the action and the type of the action, and an actual action, which is an actual amount of the action of each of the subjects, a type of the action, or a combination of the actual amount of the action and the type of the action; and
training a state prediction model, for each type related to the action, based on a lifelog of each of the subjects classified by the type, wherein
the state prediction model is obtained by performing machine learning of a relationship between a lifelog and a predicted index value of a state to be predicted of a person for which the lifelog has been measured.

In an example aspect of the present disclosure, there is provided a program executed by a computer, the program causing the computer to:
determine a type of each of subjects related to an action based on a recommended action, which is an amount of the action recommended to each of the subjects, a type of the action, or a combination of the amount of the action and the type of the action, and an actual action, which is an actual amount of the action of each of the subjects, a type of the action, or a combination of the actual amount of the action and the type of the action; and
train a state prediction model, for each type related to the action, based on a lifelog of each of the subjects classified by the type, wherein
the state prediction model is obtained by performing machine learning of a relationship between a lifelog and a predicted index value of a state to be predicted of a person for which the lifelog has been measured.

An example advantage according to the present disclosure is to train a model for highly accurately predicting a state of a subject or perform state prediction using the model.

### BRIEF DESCRIPTION OF THE DRAWINGS

FIG. 1 illustrates a schematic configuration of a state prediction system;

FIG. 2A illustrates a hardware configuration of a training device; FIG. 2B illustrates a hardware configuration of a state prediction device;

FIG. 3 illustrates an outline of state prediction of a prediction subject using a state prediction model;

FIG. 4 is an example of functional blocks of the training device related to training of a state prediction model;

FIG. 5 illustrates an outline of the training of the state prediction model;

FIG. 6 illustrates a list of the state prediction models to be trained;

FIG. 7 is an example of functional blocks of the state prediction device related to prediction using the state prediction model;

FIG. 8 illustrates an outline of the state prediction using the state prediction model when A=1;

FIG. 9 is an exemplary flowchart illustrating a process of training the state prediction model;

FIG. 10 is an exemplary flowchart illustrating a process of prediction using the state prediction model;

FIG. 11 illustrates a schematic configuration of the state prediction system;

FIG. 12 is a block diagram of the training device;

FIG. 13 is an exemplary flowchart to be executed by the training device;

FIG. 14 is a block diagram of the state prediction device; and

FIG. 15 is an exemplary flowchart to be executed by the state prediction device.

### EXAMPLE EMBODIMENT

Hereinafter, example embodiments of a training device, a state prediction device, a training method, and a program will be described with reference to the drawings.

### First Example Embodiment

### (1) System Configuration

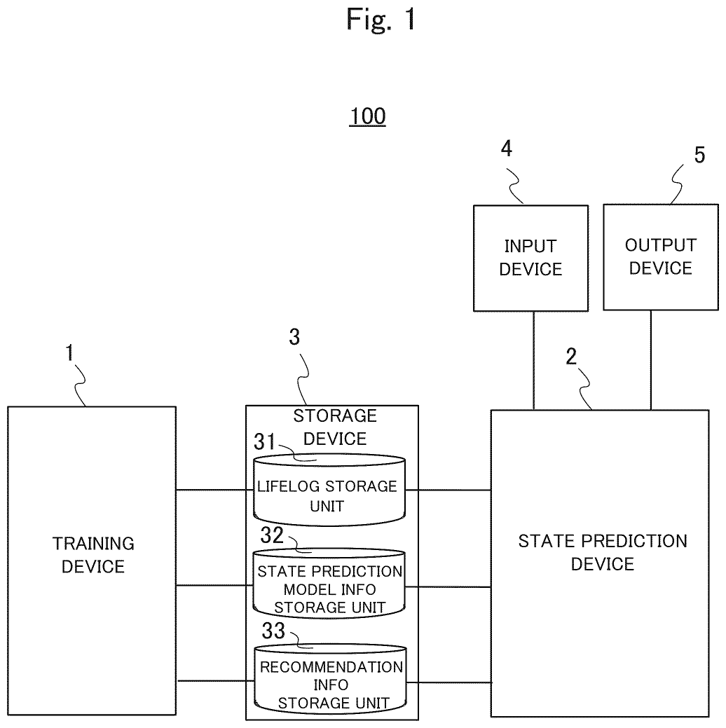

FIG. 1 illustrates a schematic configuration of a state prediction system 100. The state prediction system 100 is a system related to health management of a subject, and trains (indicating machine learning, the same applies hereinafter) a state prediction model for predicting a state of the subject from a lifelog, which is history information regarding at least one of life, behavior, and experience of the subject. The state prediction system 100 further performs state prediction using the state prediction model obtained by the training, and recommends action amount to be performed by the subject using a recommendation model trained in advance.

Hereinafter, the “subject” may be a person whose lifelog is collected, who may be a person whose behavior is managed by an organization, or may be an individual user. For example, the subject may be a patient, and may be a person whose healthcare is managed by a doctor, a nurse, or other medical worker. Hereinafter, the subject whose lifelog to be used for the training of the state prediction model is measured will particularly be referred to as a “training subject”, and the subject whose state is to be predicted by a state prediction device 2 will particularly be referred to as a “prediction subject”. The “state” to be predicted (i.e., type of the state to be predicted) is represented by a predetermined index, and examples of such an index include a body weight, a blood glucose level, a blood pressure, and any other index serving as a rough indication of health management. The items related to the state to be predicted are included in the lifelog. The “action amount” recommended to the subject is, for example, calories burned per unit period recommended to be burned by exercise, and may be represented by any other index indicating an amount of action of the subject.

The state prediction system 100 mainly includes a training device 1, the state prediction device 2, a storage device 3, an input device 4, and an output device 5. Here, the training device 1 and the storage device 3, and the state prediction device 2 and the storage device 3 perform data communication via a communication network or by wired or wireless direct communication. Likewise, the state prediction device 2 and the input device 4, and the state prediction device 2 and the output device 5 perform data communication via a communication network or by wired or wireless direct communication.

The training device 1 performs machine learning of the state prediction model based on the lifelog stored in a lifelog storage unit 31 of the storage device 3, and stores parameters of the state prediction model obtained by the machine learning in a state prediction model information storage unit 32 of the storage device 3.

The state prediction device 2 predicts a state of the prediction subject whose state is managed and determines an action amount to be recommended (which will also be referred to as a “recommended action amount”), and presents information regarding a state prediction result and determined recommended action amount to the prediction subject. In the present example embodiment, a target deadline, which is a deadline for the state management of the prediction subject, is set. Then, the state prediction device 2 determines a state at the target deadline predicted based on the lifelog and a recommended action amount. Hereinafter, it is assumed that the recommended action amount indicates an action amount per unit period (e.g., per day or per week). The target deadline corresponds to a predicted time point at which the state is predicted.

In the state prediction, the state prediction device 2 generates a state prediction model based on the parameters stored in the lifelog storage unit 31 of the storage device 3, and predicts a state of the prediction subject based on the generated state prediction model and the lifelog of the prediction subject. The state prediction device 2 further generates a recommendation model based on the parameters stored in the recommendation information storage unit 33, and determines a recommended action amount based on the generated recommendation model and the lifelog of the prediction subject. Then, the state prediction device 2 causes the output device 5 to output information regarding the predicted state of the prediction subject and the recommended action amount. In this case, the state prediction device 2 generates an output signal related to at least one of display or sound, supplies the generated output signal to the output device 5, and causes the output device 5 to display the information described above. As a result, the state prediction device 2 presents information required for the state management to the prediction subject or the like, and suitably supports the decision making of the prediction subject related to the state management. The state prediction device 2 may receive, for example, designation regarding a state to be predicted (which may be of any type, such as body weight, blood glucose level, blood pressure, etc.) and the target deadline based on an input signal supplied from the input device 4. In that case, at least one of the designated state to be predicted and the target deadline may be used as a parameter of the state prediction model and recommendation model.

The input device 4 is an interface that receives manual input (external input) of information regarding the prediction subject. A user who inputs the information using the input device 4 may be the prediction subject him/herself, or may be a person who manages or takes control of the activity of the prediction subject. The input device 4 may be, for example, various types of user input interfaces, such as a touch panel, a button, a keyboard, a mouse, an audio input device, or the like. The input device 4 supplies a generated input signal S1 to the state prediction device 2. The output device 5 displays or outputs sound of predetermined information based on an output signal S2 supplied from the state prediction device 2. Examples of the output device 5 include a display, a projector, and a speaker.

The storage device 3 is a memory that stores various types of information required for processing to be executed by the training device 1 and the state prediction device 2. The storage device 3 may be an external storage device, such as a hard disk connected to or incorporated in one of the training device 1 and the state prediction device 2, or may be a storage medium, such as a portable flash memory. The storage device 3 may be a server device that performs data communication with the training device 1 and the state prediction device 2. The storage device 3 may include multiple devices.

The storage device 3 functionally includes the lifelog storage unit 31, the state prediction model information storage unit 32, and the recommendation information storage unit 33.

The lifelog storage unit 31 stores the lifelog of the training subject and the prediction subject. As the lifelog of each subject, for example, information regarding multiple items related to life, behavior, and experience is stored in the lifelog storage unit 31 in association with date and time information of measurement or input, identification information of the subject, and the like. Examples of the items described above include gender, age, height, menstrual period (only women), body weight, body-fat percentage, number of steps, exercise item name, time, calories burned per day, number of items by mealtime, total calories taken in at mealtime, meal record registration time, daily sleep hours, blood pressure (systolic blood pressure and diastolic blood pressure), and body temperature. Those items are stored in the lifelog storage unit 31 as quantified index values. For a value of an item that changes over time, for example, an average value (or another representative value) per unit period (e.g., per week) is calculated, and the calculated average value is used for the training or prediction. The lifelog includes at least an item required to determine a recommended action amount, an item related to a state to be predicted, and an item serving as an input for the state prediction.

The lifelog may be data measured by a sensor that measures each subject, or may be data input by each subject or a manager thereof. The sensor described above may be provided in the state prediction device 2, and the lifelog of the prediction subject based on a measurement signal output from the sensor may be supplied to the lifelog storage unit 31 by the state prediction device 2. The sensor may be a wearable terminal worn by the subject, a camera that images the subject, a microphone or the like that generates audio signals of utterances of the subject, or a terminal operated by the subject, such as a personal computer, a smartphone, or the like. In that case, the state prediction device 2 may be the terminal such as a personal computer, a smartphone, or the like described above. The wearable terminal, the smartphone, and the like described above include, for example, a global navigation satellite system (GNSS) receiver, an acceleration sensor, a sensor that detects other biological signals, and the like, and the lifelog may be generated based on output signals of those sensors.

The state prediction model information storage unit 32 stores parameters of the state prediction model trained by the training device 1. The parameters of each state prediction model stored in the state prediction model information storage unit 32 are generated and updated by the training device 1. The state prediction model is a model that learns a relationship between a lifelog and a predicted index value of a state to be predicted regarding a person whose lifelog has been measured. The state prediction model is trained in such a way that, when the lifelog is input to the state prediction model, a prediction result of the state of the person whose lifelog has been measured (i.e., predicted index value of the state to be predicted) is output. As will be described later, a plurality of state prediction models according to a type is trained using the lifelog classified according to the type of the training subject. The type described above is classified based on whether the training subject has behaved in accordance with (i.e., in conformity with) the recommendation result by the recommendation model. A specific example of a case where the state prediction model is a linear model will be described later.

The state prediction model is not limited to a linear model, and may be a deep learning model based on a neural network or another machine learning model (including a statistical model). When a model based on a neural network is used, the state prediction model information storage unit 32 stores information regarding various parameters, such as a layer structure adopted in the model, a neuron structure of each layer, the number of filters and a filter size in each layer, a weight of each element of each filter, and the like.

The recommendation information storage unit 33 stores parameters of the trained recommendation model. When an index value of a predetermined item included in the lifelog is input, the recommendation model is trained in such a way that the recommended action amount of the person whose lifelog has been measured is output. For example, a plurality of recommendation models is prepared depending on the number of days (number of weeks) until the target deadline. For example, when the state to be managed is a body weight, the recommendation model outputs a recommended action amount estimated to be required to meet a weight reduction goal after a predetermined number of weeks (different for each recommendation model) from a time point of a state management start, which is a start of activity of lifestyle improvement. The recommendation model may be, for example, a linear model to be described as an exemplary state prediction model later, or may be a deep learning model based on a neural network or another machine learning model (including a statistical model). The recommended action amount may be determined using a lookup table or the like instead of being calculated using a machine learning model. In that case, for example, the lookup table indicates a correspondence relationship between a recommended action amount and an index value that may be derived by the lifelog.

The recommendation information storage unit 33 stores the recommended action amount of the prediction subject output from the recommendation model in association with the date and time information of execution of the recommendation model. The stored recommended action amount is used to determine a type based on whether the prediction subject has complied with the recommended action amount in the next state prediction of the prediction subject.

The configuration of the state prediction system 100 illustrated in FIG. 1 is an example, and various modifications may be made to the configuration. For example, at least two of the training device 1, the state prediction device 2, and the storage device 3 may be implemented by the same device. As another example, the training device 1 and the state prediction device 2 may each include a plurality of devices. In that case, the plurality of devices included in the training device 1 and the plurality of devices included in the state prediction device 2 exchange information required to execute pre-assigned processing between the devices by wired or wireless direct communication or by communication via a network. In that case, the training device 1 functions as a training system, and the state prediction device 2 functions as a state prediction system. As still another example, the input device 4 and the output device 5 may be configured in an integrated manner. In that case, the input device 4 and the output device 5 may be configured as a tablet terminal integrated with or separated from the state prediction device 2.

### (2) Hardware Configuration

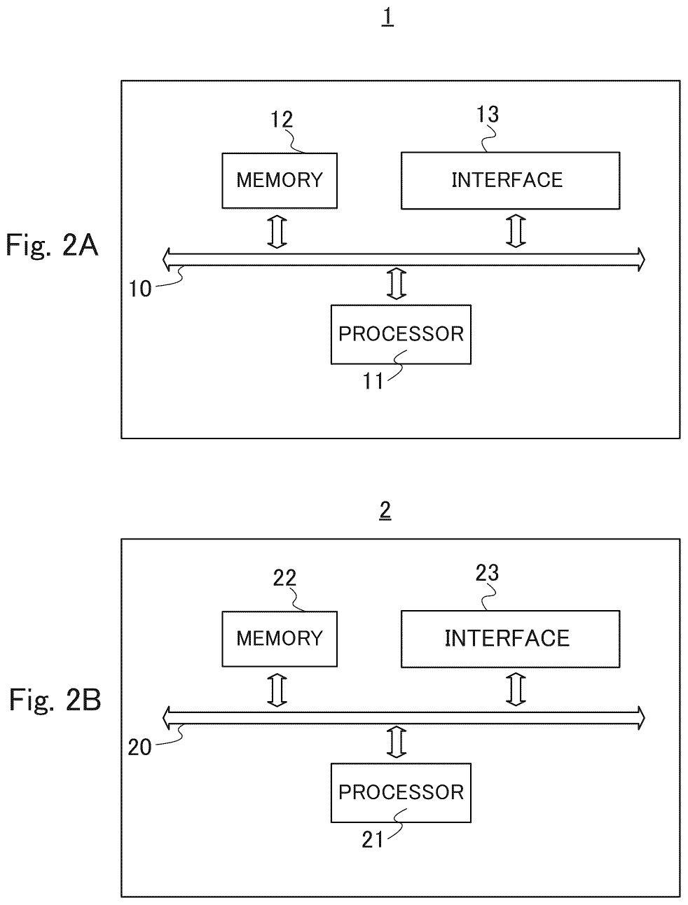

FIG. 2A illustrates a hardware configuration of the training device 1. The training device 1 includes, as hardware, a processor 11, a memory 12, and an interface 13. The processor 11, the memory 12, and the interface 13 are coupled to each other via a data bus 10.

The processor 11 functions as a controller (arithmetic device) that takes overall control of the training device 1 by executing a program stored in the memory 12. Examples of the processor 11 include a central processing unit (CPU), a graphics processing unit (GPU), and a tensor processing unit (TPU). The processor 11 may include a plurality of processors. The processor 11 is an exemplary computer.

The memory 12 includes various volatile memories, such as a random access memory (RAM), a read only memory (ROM), a flash memory, and the like, and a nonvolatile memory. The memory 12 stores a program for executing processing to be performed by the training device 1. A part of the information stored in the memory 12 may be stored in one or more external storage devices capable of communicating with the training device 1, or may be stored in a storage medium detachable from the training device 1.

The interface 13 is an interface for electrically connecting the training device 1 and another device. Those interfaces may be a wireless interface such as a network adapter for wirelessly exchanging data with another device, or may be a hardware interface for connecting to the another device by a cable or the like.

The hardware configuration of the training device 1 is not limited to the configuration illustrated in FIG. 2A. For example, the training device 1 may further include a display unit such as a display, an input unit such as a keyboard and a mouse, an audio output unit such as a speaker, and the like.

FIG. 2B illustrates an exemplary hardware configuration of the state prediction device 2. The state prediction device 2 includes, as hardware, a processor 21, a memory 22, and an interface 23. The processor 21, the memory 22, and the interface 23 are coupled to each other via a data bus 20.

The processor 21 functions as a controller (arithmetic device) that takes overall control of the state prediction device 2 by executing a program stored in the memory 22. The processor 21 is, for example, a processor such as a CPU, a GPU, a TPU, a quantum processor, or the like. The processor 21 may include a plurality of processors. The processor 21 is an exemplary computer.

The memory 22 includes various volatile memories, such as a RAM, a ROM, a flash memory, and the like, and a nonvolatile memory. The memory 22 stores a program for executing processing to be performed by the state prediction device 2. A part of the information stored in the memory 22 may be stored in an external storage device, such as the storage device 3 capable of communicating with the state prediction device 2, or may be stored in a storage medium detachable from the state prediction device 2. The memory 22 may store the information stored in the storage device 3 instead.

The interface 23 is an interface for electrically connecting the state prediction device 2 and another device. Those interfaces may be a wireless interface such as a network adapter for wirelessly exchanging data with another device, or may be a hardware interface for connecting to the another device by a cable or the like.

The hardware configuration of the state prediction device 2 is not limited to the configuration illustrated in FIG. 2B. For example, the state prediction device 2 may incorporate any of the input device 4 and the output device 5 instead of being connected to them via the interface 23.

### (3) Outline of State Prediction

An outline of the state prediction using the state prediction model will be described. Hereinafter, it is assumed that the state prediction model predicts a state based on the lifelog for at least the latest “A” (A is an integer of 1 or more) weeks.

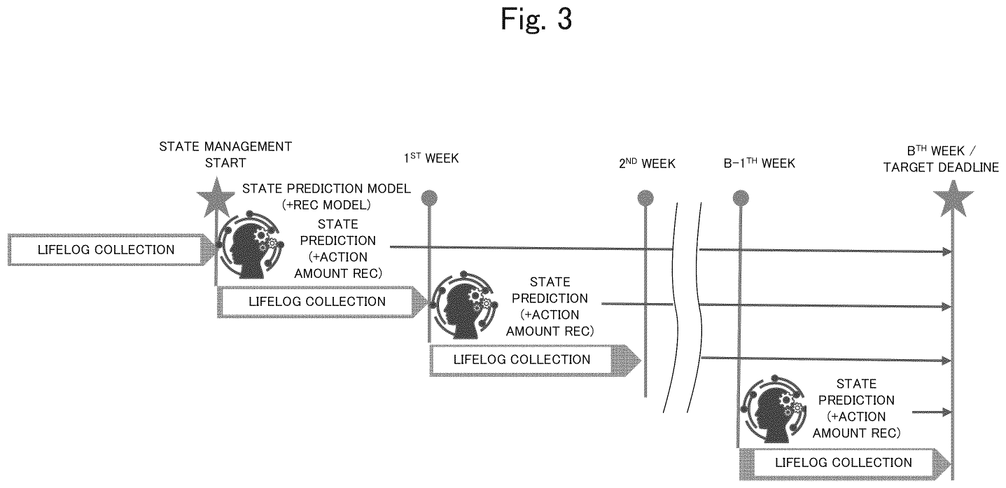

FIG. 3 illustrates an outline of the state prediction of the prediction subject using the state prediction model. FIG. 3 illustrates an outline of the state prediction up to a target deadline after “B” (B is an integer of equal to or more than 2) weeks from the start of the state management of the prediction subject. Hereinafter, as an example, it is assumed that the state prediction and the recommendation of the action amount are executed every week from the start of the state management to the “B−1”-th week.

As illustrated in FIG. 3, the lifelog of the prediction subject is collected before the start of the state management, and the state prediction device 2 predicts, at the time point of the state management start, a state of the prediction subject at the target deadline, which is after B weeks, based on the collected lifelog and the state prediction model. In this case, the state prediction model used at the time point of the state management start is a model that outputs a prediction result of the state after B weeks based on at least the lifelog for the latest A weeks before the start of the state management. The state prediction device 2 further calculates a recommended action amount for the prediction subject until the target deadline after B weeks based on the collected lifelog and the recommendation model. Here, the state prediction device 2 may calculate a recommended action amount for the next week from the time point of the state management start, which is optimal for the prediction subject to meet the goal by the target deadline after B weeks. The state prediction device 2 presents the state prediction result and the recommended action amount to the prediction subject or the manager thereof.

In the first week from the start of the state management, the state prediction device 2 predicts the state of the prediction subject by the target deadline after “B−1” weeks again based on the lifelog for the latest A weeks and the state prediction model. The state prediction model used in this case is a model that outputs a prediction result of the state after “B−1” weeks based on at least the lifelog for the latest A weeks. As will be described later, in this case, the state prediction device 2 determines a type of the prediction subject based on whether the prediction subject has complied with the recommended action amount calculated at the time point of the state management start, and predicts a state of the prediction subject using the state prediction model depending on the determined type. The state prediction device 2 further calculates a recommended action amount for the prediction subject until the target deadline after “B−1” weeks based on the collected lifelog and the recommendation model. Here, the state prediction device 2 may calculate a recommended action amount for the next week from the time point of the first week from the state management start, which is optimal for the prediction subject to meet the goal by the target deadline after “B−1” weeks. The state prediction device 2 presents the state prediction result and the recommended action amount to the prediction subject or the manager thereof.

Then, the state prediction device 2 executes the prediction of the state and the recommendation of the action amount described above every week, and in the “B−1”-th week from the start of the state management, the state prediction device 2 predicts a state of the prediction subject at the target deadline after one week based on the lifelog for the latest A weeks and the state prediction model. The state prediction model used in this case is a model that outputs a prediction result of the state after one week based on at least the lifelog for the latest A weeks. In this case, the state prediction device 2 determines a type of the prediction subject based on whether the prediction subject has complied with the recommended action amount output in the “B−2”-th week, and predicts a state of the prediction subject using the state prediction model depending on the determined type. The state prediction device 2 further calculates a recommended action amount for the prediction subject until the target deadline after one week based on the collected lifelog and the recommendation model. The state prediction device 2 presents the state prediction result and the recommended action amount to the prediction subject or the manager thereof.

Here, a problem in a case where both the state prediction and the recommendation of the action amount are performed will be described in a supplemental manner. The state prediction using the state prediction model and the recommendation of the action amount using the recommendation model are executed by different models. Thus, even if the prediction subject acts as recommended, the state prediction result may indicate an unimproved state when the state prediction using the state prediction model and the recommendation of the action amount using the recommendation model are independently executed. On the other hand, the state prediction result may indicate an improved state even if the subject does not act as recommended. In that case, the user may be less motivated to comply with the behavior change, and the user may stop using the present system.

In view of the above, the state prediction system 100 according to the present example embodiment trains a state prediction model depending on the type classified based on whether the recommendation by the recommendation model has been complied with, and predicts a state using the state prediction model. As a result, the state prediction system 100 is enabled to train a state prediction model capable of highly accurate state prediction and to predict a state using the state prediction model.

### (4) Training Phase of State Prediction Model

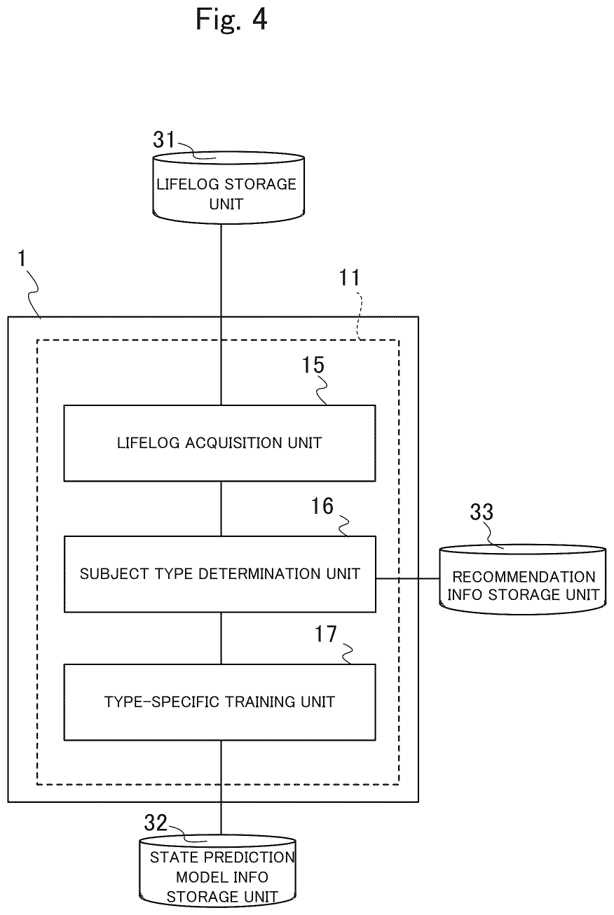

FIG. 4 is an example of functional blocks of the training device 1 related to the training of the state prediction model. The processor 11 of the training device 1 functionally includes a lifelog acquisition unit 15, a subject type determination unit 16, and a type-specific training unit 17. While blocks that exchange data with each other are connected by a solid line in FIG. 4, a combination of the blocks that exchange data with each other is not limited thereto. Other functional block diagrams to be described later are in a similar manner. While descriptions will be given on the assumption that the state prediction is performed on a weekly basis hereinafter for explanatory convenience, it is not limited thereto, and the state prediction may be performed on a daily basis or a monthly basis, or the state prediction may be performed per predetermined number of days.

Here, training of a state prediction model that predicts a state after “X” (X is an integer satisfying 1≤X≤B) weeks will be described. The sign “X” corresponds to a time length (i.e., predicted time length) from the prediction time point using the state prediction model to the target deadline. In FIG. 3, the state prediction model used at the time point of the state management start corresponds to “X=B”, the state prediction model used at the first week corresponds to “X=B−1”, and the state prediction model used at the “B−1”-th week corresponds to “X=1”.

The lifelog acquisition unit 15 obtains, from the lifelog storage unit 31, the lifelog of the training subject to be used to train the state prediction model. In this case, the lifelog acquisition unit 15 determines, in the past acquisition period of the lifelog of each training subject stored in the lifelog storage unit 31, a time point to be associated with the state management start (state management start time point on the log) and a time point to be associated with the target deadline (target deadline on the log). The lifelog acquisition unit 15 further determines a time point to be associated with the prediction time point (prediction time point on the log) at which the prediction is carried out using the state prediction model. Then, the lifelog acquisition unit 15 obtains the lifelog required to input the state prediction model. For example, when the state prediction model is a linear model to be described later, the lifelog for X weeks between the prediction time point on the log and the target deadline on the log, and the lifelog for A weeks immediately before the prediction time point on the log are obtained for each training subject. The linear model described above may be a model that predicts a state using only the lifelog for A weeks immediately before the prediction time point on the log. In that case, the lifelog acquisition unit 15 obtains the lifelog for A weeks immediately before the prediction time point on the log for each training subject. The lifelog acquisition unit 15 obtains, from the lifelog storage unit 31, the index value representing the state to be predicted included in the lifelog at the target deadline on the log representing the state of the correct answer to be output by from the state prediction model. In addition to the lifelog described above, the lifelog acquisition unit 15 may extract, from the lifelog storage unit 31, the lifelog required for the subject type determination unit 16 to execute the recommendation model. The lifelog acquisition unit 15 supplies the obtained lifelog to the subject type determination unit 16.

The subject type determination unit 16 determines a type of the training subject whose lifelog supplied from the lifelog acquisition unit 15 has been measured. Here, the type of the training subject is classified based on whether the training subject has acted in conformity with the recommended action amount. Hereinafter, a “first type” represents a subject who has acted in conformity with the recommended action amount (i.e., recommendation-complying person). A “second type” represents a subject who has not acted in conformity with the recommended action amount (i.e., non-recommendation-complying person). A “third type” represents a subject who is unclear whether the subject has acted in conformity with the recommended action amount (i.e., person with unknown propensity).

A specific example of the type determination method in this case will be described. The subject type determination unit 16 determines the type described above based on a result of comparison between the recommended action amount at the time point one week before the prediction time point on the log and the observed action amount (which will also be referred to as an “actual action amount”) in a period calculated from the lifelog of the period from the time point one week before to the prediction time point on the log. In this case, the subject type determination unit 16 inputs the lifelog obtained before the time point one week before the prediction time point on the log to the recommendation model generated by referring to the recommendation information storage unit 33, and obtains the recommended action amount output from the recommendation model. The subject type determination unit 16 calculates the actual action amount from the lifelog of the period from the time point one week before the prediction time point on the log to the prediction time point on the log. For example, when the lifelog includes information regarding calories burned, the actual action amount is calculated based on the information. The subject type determination unit 16 may calculate the actual action amount from the lifelog in accordance with any calculation method. When the recommended action amount described above is actually recommended to the training subject and the recommended action amount is stored in the recommendation information storage unit 33 or the like, the subject type determination unit 16 may determine a type by using the stored recommended action amount without executing the recommendation model. Likewise, when the actual action amount described above is stored in the recommendation information storage unit 33 or the like, the subject type determination unit 16 may determine a type by using the stored actual action amount instead of calculating the actual action amount from the lifelog.

Then, the subject type determination unit 16 determines that the training subject whose recommended action amount and actual action amount are within a predetermined difference is the first type, and determines that the training subject whose recommended action amount and actual action amount are larger than the predetermined difference is the second type. For example, the predetermined difference described above is set to a predetermined value stored in the storage device 3, the memory 12, or the like. Meanwhile, the subject type determination unit 16 determines that the training subject whose information for calculating the actual action amount is insufficient in the lifelog is the third type. It is assumed that the recommended action amount and the actual action amount represent an action amount (calories, etc.) per common unit period (e.g., per day).

Then, the subject type determination unit 16 supplies, to the type-specific training unit 17, the determined type of each training subject and the lifelog of each training subject. In the case of “X=B”, that is, in the case of training a state prediction model to be used at the time point of the state management start, a common state prediction model is used regardless of the type, and thus the subject type determination unit 16 supplies, to the type-specific training unit 17, the lifelog obtained by the lifelog acquisition unit 15 without determining the type.

The type-specific training unit 17 trains a state prediction model depending on the type of the training subject determined by the subject type determination unit 16. Specifically, the type-specific training unit 17 trains a state prediction model for the first type using the lifelog of the training subject determined to be the first type. The type-specific training unit 17 further trains a state prediction model for the second type using the lifelog of the training subject determined to be the second type, and trains a state prediction model for the third type using the lifelog of the training subject determined to be the third type. Here, when the number of training subjects determined to be the third type is equal to or less than a predetermined number, the type-specific training unit 17 may train the state prediction model for the third type using all the lifelogs of the training subjects of the first type and the second type. Likewise, when the number of training subjects determined to be the first type is equal to or less than a predetermined number, the type-specific training unit 17 may train the state prediction model for the first type using all the lifelogs of the training subjects. Likewise, when the number of training subjects determined to be the second type is equal to or less than a predetermined number, the type-specific training unit 17 may train the state prediction model for the second type using all the lifelogs of the training subjects. In the case of “X=B”, that is, in the case of training the state prediction model to be used at the time point of the state management start, the type-specific training unit 17 trains a common state prediction model not dependent on the type of the training subject based on the lifelog obtained by the lifelog acquisition unit 15. Then, the type-specific training unit 17 stores the parameters of the state prediction model obtained through the training in the state prediction model information storage unit 32.

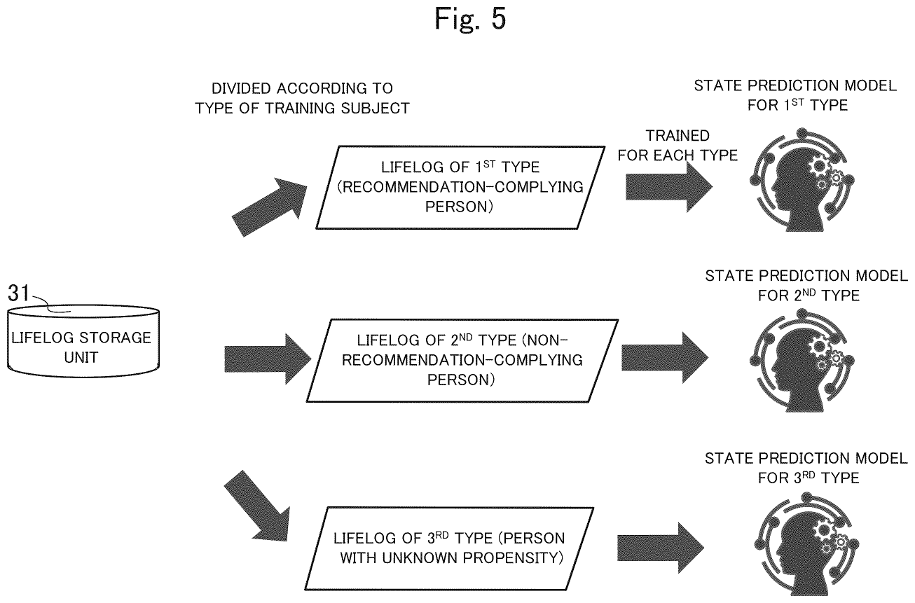

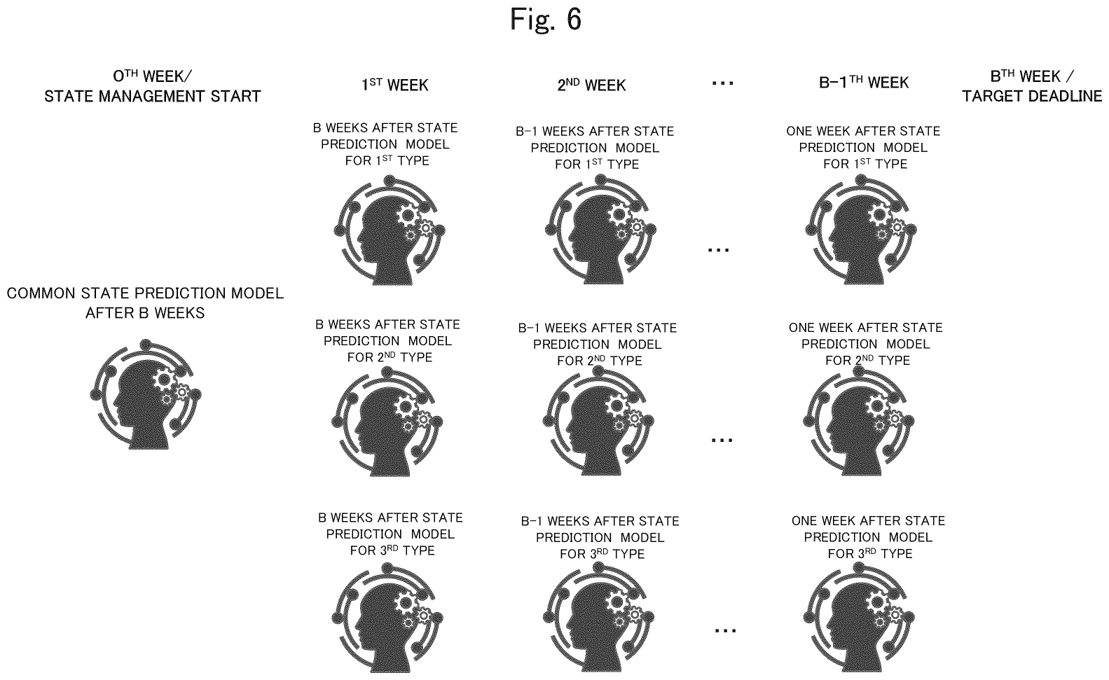

FIG. 5 illustrates an outline of the training of the state prediction model by the type-specific training unit 17. As illustrated in FIG. 5, the lifelog extracted from the lifelog storage unit 31 is divided depending on the type of the training subject. Here, it is divided into the lifelog of the first type, the lifelog of the second type, and the lifelog of the third type. Then, the type-specific training unit 17 trains a state prediction model from the lifelog for each type. As a result, the state prediction model for the first type, the state prediction model for the second type, and the state prediction model for the third type are trained. As will be described in detail with reference to FIG. 6 to be described later, the type-specific training unit 17 trains a prediction model for each time point of X=1 to B.

Here, the training in the case where the state prediction model is a linear model will be described in a supplemental manner.

When the state prediction model is a linear model, a state to be predicted (body weight, etc.) “y” is expressed as follows using a variable vector “x” of the lifelog for “A+X” weeks.

$$y = cx + d$$

Here, “c” represents a vector of the same dimension as x, and “d” represents a coefficient. Each element of the vector c and the coefficient d are equivalent to parameters to be obtained through the training.

When there are “f” indexes (i.e., indexes to be used for the state prediction) relevant to the items to be used for the prediction among the items included in the lifelog for one week, the vector x is a vector having a length of “(f+1)×A+f×X)”. The “indexes to be used for the state prediction” may be any indexes other than an index representing the state to be predicted itself. For example, in a case of predicting a body weight, indexes other than the body weight (calories burned, sleep hours, blood pressure, body temperature, etc.) are used as the “indexes to be used for the state prediction”. Then, for each training subject, the type-specific training unit 17 sets “x” based on the lifelog for A weeks immediately before the prediction time point on the log and the lifelog for X weeks from the prediction time point on the log, and sets “y” based on the lifelog for one week between the time point after X−1 weeks from the prediction time point on the log and the time point after X weeks from the prediction time point on the log. For example, when “y” represents a body weight, the type-specific training unit 17 may set, as “y”, an average value of the values of the body weight for one week between the time point after X−1 weeks from the prediction time point on the log and the time point after X weeks from the prediction time point on the log. Then, the type-specific training unit 17 obtains the vector c and the coefficient d by any approximate method using at least equal to or more than the number of parameters to be obtained (i.e., (f+1)×A+f×X+1, which is the total number of the number of elements of c and the coefficient d) of sets of the lifelog for “A+X” weeks and the lifelog after X weeks. Then, the type-specific training unit 17 stores the parameters of the state prediction model obtained for each type in the state prediction model information storage unit 32.

FIG. 6 illustrates a list of the state prediction models to be trained by the type-specific training unit 17. As illustrated in FIG. 6, the type-specific training unit 17 trains one state prediction model to be used at the time point of the state management start (i.e., state prediction model satisfying “X=B”), and trains a state prediction model to be used in the first week from the state management start (i.e., state prediction model satisfying “X=B−1”) for each of the first type to the third type. Likewise, the type-specific training unit 17 trains a state prediction model to be used in each of the second week to the “B−1”-th week from the state management start for each of the first type to the third type.

In this manner, the lifelog acquisition unit 15, the subject type determination unit 16, and the type-specific training unit 17 change X in the range from one week to B weeks, and sequentially perform processing for training a state prediction model to be used from the time point of the state management start to the “B−1”-th week. As a result, “3×B−2” state prediction models are trained. Then, the type-specific training unit 17 stores the parameters of those trained state prediction models in the state prediction model information storage unit 32.

Predicting a state of B weeks later using an input for “B+1” weeks in the case of “X=B” is equivalent to “predicting a value of the index value of the state to be predicted related to a week from the (B−1)-th week from the time point of the state management start to the B-th week in the case of living for B+1 weeks from one week before the time point of the state management start”. While the lifelog of the period A includes the index value of the state to be predicted after X weeks, the lifelog of the period X does not include the index value of the state to be predicted. The index value of the state to be predicted after X weeks is the prediction target.

Here, each component of the lifelog acquisition unit 15, the subject type determination unit 16, and the type-specific training unit 17 may be implemented by, for example, the processor 11 executing a program. Each component may be implemented in such a way that a required program is recorded in any nonvolatile storage medium and is installed as appropriate. At least some of those components are not limited to be implemented by software using a program, and may be implemented by, for example, a combination of any of hardware, firmware, and software. At least some of those components may be implemented using, for example, a user-programmable integrated circuit, such as a field-programmable gate array (FPGA), a microcontroller, or the like. At least some of those components may include an application specific standard produce (ASSP), an application specific integrated circuit (ASIC), a quantum processor (quantum computer control chip), or the like. As described above, the components may be implemented by various types of hardware. Other example embodiments to be described later are in a similar manner. Moreover, those components may be implemented by cooperation of multiple computers using, for example, cloud computing technology or the like.

### (5) Prediction Phase Using State Prediction Model

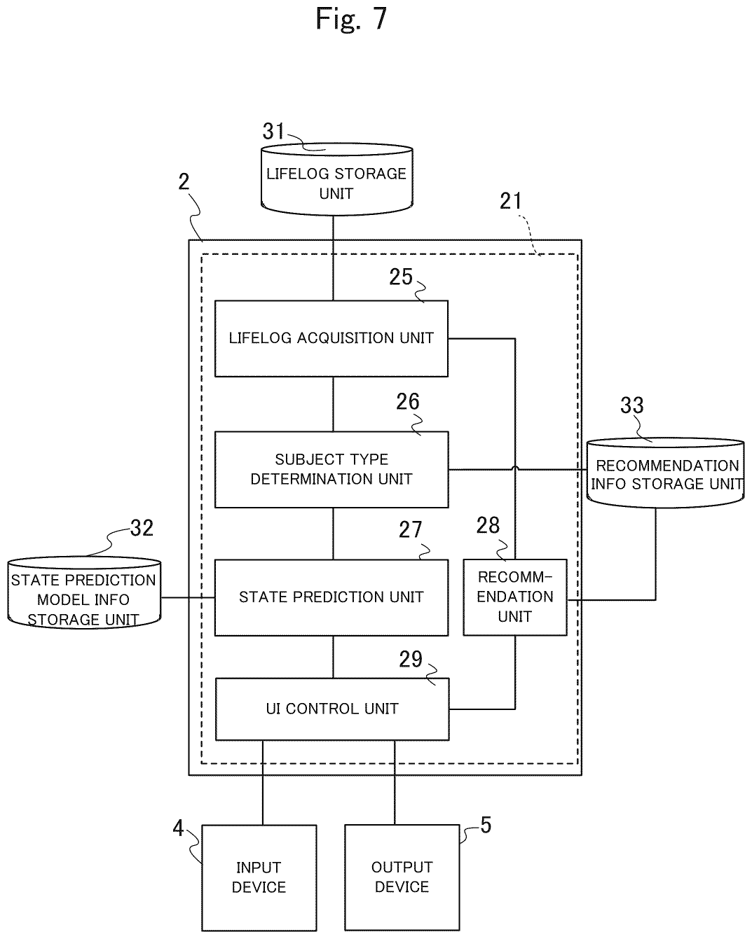

FIG. 7 is an example of functional blocks of the state prediction device 2 related to the prediction using the state prediction model. The processor 21 of the state prediction device 2 functionally includes a lifelog acquisition unit 25, a subject type determination unit 26, a state prediction unit 27, a recommendation unit 28, and a UI control unit 29. Here, a case of predicting a state after X weeks will be described. In FIG. 3, the case of the state prediction model used at the time point of the state management start corresponds to “X=B”, the case of the state prediction model used at the first week corresponds to “X=B−1”, and the case of the state prediction model used at the “B−1”-th week corresponds to “X=1”.

The lifelog acquisition unit 25 obtains the lifelog of the prediction subject from the lifelog storage unit 31. In this case, for example, the lifelog acquisition unit 25 obtains, from the lifelog storage unit 31, the lifelog for the latest A weeks required for the input to the state prediction model. Then, the lifelog acquisition unit 25 supplies, to the subject type determination unit 26, the lifelog for the latest A weeks required for the input to the state prediction model. The lifelog acquisition unit 25 further supplies, to the recommendation unit 28, the lifelog required for the subject type determination unit 26 to execute the recommendation model.

The subject type determination unit 26 determines a type of the prediction subject whose lifelog supplied from the lifelog acquisition unit 25 has been measured. In this case, the subject type determination unit 26 refers to the recommendation information storage unit 33 to obtain the recommended action amount recommended to the prediction subject in the immediately preceding week. The subject type determination unit 26 further calculates an actual action amount based on the lifelog from the immediately preceding week to the present, and determines a type of the prediction subject based on a result of comparison between the calculated actual action amount and the recommended action amount. Then, the subject type determination unit 26 determines that the prediction subject whose recommended action amount and actual action amount are within a predetermined difference is the first type, and determines that the prediction subject whose recommended action amount and actual action amount are larger than the predetermined difference is the second type. Meanwhile, the subject type determination unit 26 determines that the prediction subject whose information for calculating the actual action amount is insufficient in the lifelog is the third type. The subject type determination unit 26 supplies the lifelog and the determined type of the prediction subject to the state prediction unit 27.

In the case of “X=B”, that is, in the case where the prediction time point is the time point of the state management start, a common state prediction model is used regardless of the type, and thus the subject type determination unit 26 supplies the lifelog to the state prediction unit 27 without determining the type.

The state prediction unit 27 predicts a state of the prediction subject after X weeks. In this case, the state prediction unit 27 extracts, from the state prediction model information storage unit 32, parameters of the state prediction model depending on the type determined by the subject type determination unit 26. Then, the state prediction unit 27 obtains, from the state prediction model, a result of the prediction of the state after X weeks based on the lifelog and the state prediction model generated by the extracted parameters. As a result, an accurate prediction result of the state of prediction subject after X weeks may be obtained based on the state prediction model trained specifically for the type of the prediction subject. Then, the state prediction unit 27 supplies the prediction result of the state after X weeks to the UI control unit 29. In the case of “X=B”, that is, in the case where the prediction time point is the time point of the state management start, the state prediction unit 27 predicts the state of the prediction subject based on the lifelog and the state prediction model not dependent on the type determination result.

The recommendation unit 28 determines a recommended action amount to be newly recommended to the prediction subject. In this case, the recommendation unit 28 extracts, from the recommendation information storage unit 33, the parameters of the recommendation model whose target deadline is X weeks later, and obtains the recommended action amount from a recommendation model based on the lifelog and the recommendation model generated by the extracted parameters. Then, the recommendation unit 28 supplies the obtained recommended action amount to the UI control unit 29. The recommendation unit 28 stores, in the recommendation information storage unit 33, the obtained recommended action amount in association with identification information of the prediction subject and date and time information.

The UI control unit 29 performs control related to a user interface. For example, the UI control unit 29 causes the output device 5 to display and/or output by sound the prediction result of the state after X weeks generated by the state prediction unit 27 and the recommended action amount determined by the recommendation unit 28. The UI control unit 29 may further receive, from the input device 4, model parameters, information specifying setting of the target deadline, and the like.

Here, the state prediction by the state prediction unit 27 using the state prediction model in the case where the state prediction model is a linear model will be described in a supplemental manner.

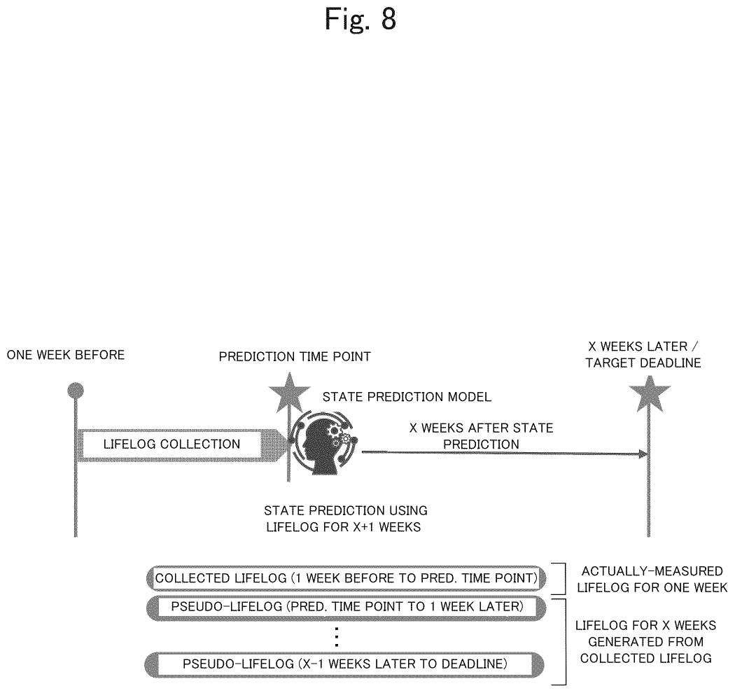

FIG. 8 illustrates an outline of the state prediction using the state prediction model in the case of “A=1”. In the case of “A=1”, the state prediction unit 27 uses the lifelog of the prediction subject actually collected in the latest one week of the prediction time point as an input to the state prediction model. As described above, when the state prediction model is a linear model described above, the lifelog for X weeks from the prediction time point to the target deadline is required in addition to the lifelog for the latest A (one in this case) weeks. In this case, the state prediction unit 27 generates a dummy lifelog (pseudo-lifelog) for X weeks from the prediction time point to the target deadline based on the lifelog (collected lifelog) for A weeks (one week in this case) collected before the prediction time point. In this case, for example, the state prediction unit 27 generates a pseudo-lifelog in which the collected lifelog is duplicated. That is, the state prediction unit 27 generates the pseudo-lifelog on the assumption that the prediction subject continues the same lifestyle as the latest one week of the prediction time point in X weeks until the target deadline. When A is equal to or more than two, the state prediction unit 27 may perform statistical processing, such as averaging, on the lifelog for the latest A weeks, and may generate the pseudo-lifelog.

Then, the state prediction unit 27 predicts a state of the prediction subject after X weeks using the state prediction model and the lifelog for “X+1” weeks obtained by combining the collected lifelog and the pseudo-lifelog. Specifically, the state prediction unit 27 generates a vector x having a length of “(f+1)×1+f×X” from the lifelog for “X+1” weeks, and obtains y, which represents the state of the prediction subject after X weeks, in accordance with “y=cx+d” using the vector c and the coefficient d obtained by training. The state prediction model may be a model that performs the state prediction using only the collected lifelog (i.e., lifelog for A weeks immediately before the prediction time point on the log) instead of performing the state prediction from the lifelog for “X+A” weeks obtained by combining the collected lifelog and the pseudo-lifelog.

Each component of the lifelog acquisition unit 25, the subject type determination unit 26, the state prediction unit 27, the recommendation unit 28, and the UI control unit 29 may be implemented by, for example, the processor 21 executing a program. Each component may be implemented in such a way that a required program is recorded in any nonvolatile storage medium and is installed as appropriate. At least some of those components are not limited to be implemented by software using a program, and may be implemented by, for example, a combination of any of hardware, firmware, and software. At least some of those components may be implemented using, for example, a user-programmable integrated circuit, such as an FPGA, a microcontroller, or the like. At least some of those components may include an ASSP, an ASIC, a quantum processor (quantum computer control chip), or the like. As described above, the components may be implemented by various types of hardware. Other example embodiments to be described later are in a similar manner. Moreover, those components may be implemented by cooperation of multiple computers using, for example, cloud computing technology or the like.

### (6) Processing Flow

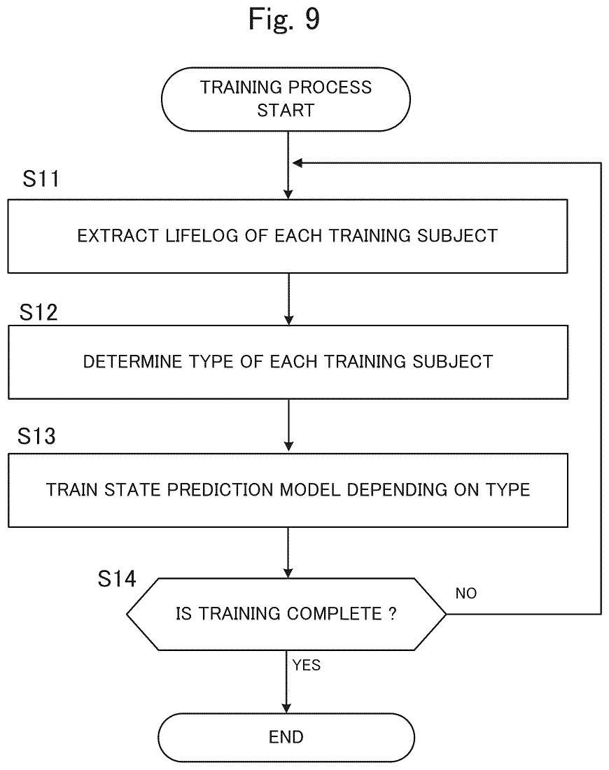

FIG. 9 is an exemplary flowchart illustrating the process of training the state prediction model to be executed by the training device 1.

First, the training device 1 extracts the lifelog of each training subject from the lifelog storage unit 31 (step S11). The extracted lifelog includes an item required to determine a recommended action amount, an item related to a state to be predicted, and an item serving as an input for the state prediction. Then, the training device 1 determines a type of each training subject (step S12). In this case, as described above, the training device 1 obtains the recommended action amount at the time point immediately before the prediction time point on the log and the actual action amount from the time point immediately before to the prediction time point, and determines a type of each training subject based on a difference between the recommended action amount and the actual action amount. Then, the training device 1 trains a state prediction model depending on the type (step S13). In this case, the training device 1 divides the lifelog into groups for each type according to the type of each training subject determined in step S12, and trains the state prediction model of the relevant type using the lifelog for each divided group. Then, the training device 1 stores parameters of the state prediction model obtained through the training in the state prediction model information storage unit 32.

Next, the training device 1 determines whether the training of the state prediction model is complete (step S14). For example, the training device 1 determines that the training is complete if the training of all the state prediction models (i.e., state prediction models of “X=1, 2, . . . , B−1, and B”) that may be used in each week from the zeroth week to the “B−1”-th week illustrated in FIG. 6 is determined to be complete. Then, if it is determined that the training is complete (Yes in step S14), the training device 1 terminates the process of the flowchart. On the other hand, if it is determined that the training is not complete (No in step S14), the training device 1 returns the process to step S11. For example, the training device 1 sequentially executes the training of the state prediction model for each X of “X=1, 2, . . . , B−1, and B” in steps S11 to S13, and executes B times of steps S11 to S13, thereby training all the state prediction models. If the state prediction model in the case of “X=B” is to be trained, the training device 1 does not execute step S12, and executes, in step S13, training of the state prediction model using the lifelog obtained in step S11.

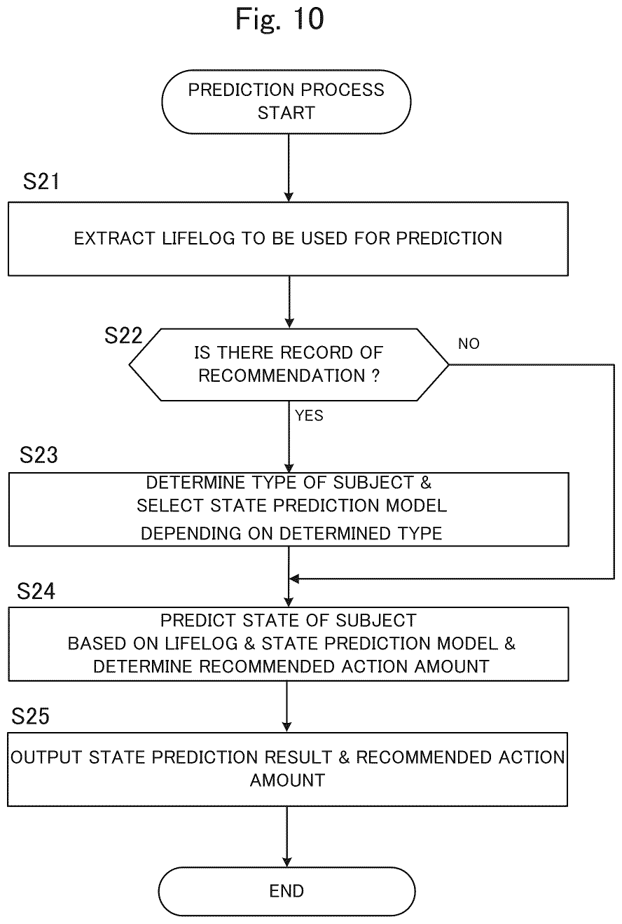

FIG. 10 is an exemplary flowchart illustrating the process of prediction using the state prediction model to be executed by the state prediction device 2. The state prediction device 2 executes the process of the flowchart illustrated in FIG. 10 when, for example, the timing is reached at which the state of the prediction subject is predicted and the action amount is recommended. The timing at which the state of the prediction subject is predicted and the action amount is recommended may be designated by a user input from the input device 4, or may be timing at which a preset date and time is reached.

First, the state prediction device 2 extracts the lifelog of the prediction subject from the lifelog storage unit 31 (step S21). Next, the state prediction device 2 determines whether there is a record of action amount recommendation to the prediction subject (step S22). Then, if there is a record of action amount recommendation to the prediction subject (Yes in step S22), the state prediction device 2 determines a type of the prediction subject, and selects the state prediction model depending on the determined type (step S23). In this case, as described above, the state prediction device 2 obtains the recommended action amount recommended immediately before the prediction time point and the actual action amount, which is the action amount observed until the prediction time point from the recommendation, and determines the type of the prediction subject based on the difference between the recommended action amount and the actual action amount, for example. Then, the state prediction device 2 extracts, from the state prediction model information storage unit 32, the parameters of the state prediction model relevant to the determined type, and generates a state prediction model relevant to the type of the prediction subject.

On the other hand, if there is no record of action amount recommendation to the prediction subject (No in step S22), the state prediction device 2 advances the process to step S24. In this case, the state prediction device 2 extracts, from the state prediction model information storage unit 32, the parameters of the state prediction model corresponding to “X=B”, and generates a state prediction model not dependent on the type of the prediction subject. In practice, the state prediction device 2 may assume a plurality of “X” to prepare a state prediction model of a third type depending on X, and may selectively use the state prediction model of the third type depending on X.

Next, the state prediction device 2 predicts a state of the prediction subject based on the state prediction model and the lifelog extracted in step S21 (step S24). In step S24, the state prediction device 2 determines a recommended action amount based on the lifelog and the recommendation model. Then, the state prediction device 2 outputs, from the output device 5, the state prediction result of the prediction subject and the recommended action amount (step S25).

### (7) Modified Examples

Next, modified examples suitable for the example embodiment described above will be described. The following modified examples may be applied in combination.

### First Modified Example

The type of the subject is not limited to being divided into three types.

For example, the training device 1 may determine the type based on, in addition to a result of the comparison between the recommended action amount and the actual action amount in the latest period, a result of the comparison between the recommended action amount and the actual action amount before that period.

For example, when X is equal to or less than “B−2”, the training device 1 may determine the type in further consideration of a result of comparison between the recommended action amount and the actual action amount in a period from two weeks before to one week before in addition to a result of the comparison between the recommended action amount and the actual action amount in the latest one week. There are nine (=3×3) patterns of combinations of the comparison results in that case. Likewise, when X is equal to or less than “B−3”, the type may be determined in further consideration of a result of the comparison between the recommended action amount and the actual action amount in a period from three weeks before to two weeks before. In those cases, the training device 1 trains a state prediction model for each type based on the lifelog divided for each type, and stores parameters of the trained state prediction model for each type in the state prediction model information storage unit 32. The state prediction device 2 also determines the type of the prediction subject by the same method as the training device 1, and selects a state prediction model to be used in the state prediction based on a result of the determination.

The training device 1 and the state prediction device 2 may classify the type of the subject in more detail based on attributes of the subject, such as gender, age, and the like.

For example, the training device 1 determines the type based on a tendency of whether to comply with the recommended action amount and the gender of the subject. In that case, the first to third types are provided for men, and the first to third types are provided for women, and thus there are six patterns of types in total. The training device 1 trains a state prediction model for each type based on the lifelog divided for each type, and stores parameters of the trained state prediction model for each type in the state prediction model information storage unit 32. In a similar manner to the training device 1, the state prediction device 2 determines a type of the prediction subject based on the tendency of whether to comply with the recommended action amount and the gender of the subject, and selects a state prediction model to be used in the state prediction based on a result of the determination. As a result, the state prediction device 2 is enabled to classify the prediction subject in more detail, and to highly accurately predict a state using the state prediction model suitable for the prediction subject.

### Second Modified Example

Although the action amount is recommended in the descriptions above, the target to be recommended is not limited to the action amount, and may be a type of action or a combination of the action amount and the type of action. In that case, the recommendation model is a model trained to output at least one of the action amount or the type of action recommended to the person whose lifelog has been measured when the index value of the predetermined item included in the lifelog is input. The recommended action amount, type of action, or combination thereof will also be referred to as a “recommended action”.

Also in the case of determining the type based on whether the subject has complied with the recommended action, the state prediction device 2 specifies an observed action amount, a type of action, or a combination thereof instead of calculating the actual action amount. Hereinafter, the observed action amount, type of action amount, and combination thereof of the subject will also be referred to as “actual action”. Then, the state prediction device 2 determines a type based on a result of comparison between the actual action of the subject and the recommended action. Here, in the case of using the type of action, it may be suitable if a state where the type of the actual action and the type of the recommended action at least match with each other is set as a necessary condition or necessary and sufficient condition for a recommendation-complying person.

Also in the present modified example, the state prediction device 2 is enabled to accurately determine the type based on, for example, whether the subject is a recommendation-complying person, and to highly accurately predict the state of the prediction subject using the state prediction model depending on the determined type.

### (8) Application Examples

The state prediction device 2 may recommend a predetermined action to the prediction subject or automatically perform a predetermined action, such as placing an online order, on behalf of the prediction subject based on the state prediction result of the prediction subject.

In this case, the state prediction device 2 may determine, based on the state prediction result, a health food product (including diet product, low calorie food, and supplement) recommended to the prediction subject, and may present the determined health food product to the prediction subject or place an online order using electronic commerce. The state prediction device 2 may present, to the prediction subject, a facility (including nearby health club) recommended to the prediction subject instead of the health food product, or may present recommended stretching or exercise to the prediction subject. The state prediction device 2 may add processing of automatically making a tentative reservation in a time period during which a facility is open on the weekend from information regarding availability of the recommended facility (nearby gymnastic hall or facility for a specific sport) and information regarding a recommended sport. Then, the state prediction device 2 may add processing of canceling the tentative reservation if the prediction subject has not made a formal reservation within a predetermined period of time. Instead of outputting those pieces of recommendation information from the output device 5, the state prediction device 2 may cooperate with a system of a facility that handles health food products, a traffic system, or the like to display recommended products or the like on a display, such as digital signage of those systems. In this case, the state prediction device 2 may specify the position of the prediction subject who is walking by performing personal authentication of pedestrians using images generated by a camera provided in those systems, and may display information on a display near the specified position of the prediction subject. The state prediction device 2 may give an incentive, such as a point that may be used by the prediction subject, when the prediction subject takes the recommended action.

### Second Example Embodiment

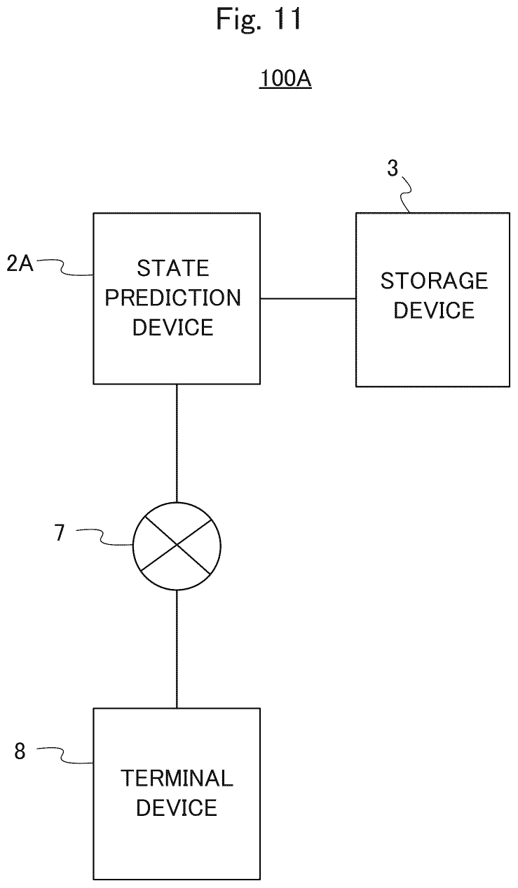

FIG. 11 illustrates a schematic configuration of a state prediction system 100A. The state prediction system 100A according to a second example embodiment is a system of a server-client model, and a state prediction device 2A, which functions as a server device, performs processing of the training device 1 and the state prediction device 2 in the first example embodiment. Hereinafter, components same as those in the first example embodiment will be denoted by the same reference signs as appropriate, and descriptions thereof will be omitted.

As illustrated in FIG. 11, the state prediction system 100A mainly includes the state prediction device 2A that functions as a server, a storage device 3 that stores data similar to that of the first example embodiment, and a terminal device 8 that functions as a client. The state prediction device 2A and the terminal device 8 perform data communication via a network 7.

The terminal device 8 is a terminal having an input function, a display function, and a communication function, and functions as the input device 4 and the output device 5 illustrated in FIG. 1. The terminal device 8 may be, for example, a personal computer, a tablet terminal, a personal digital assistant (PDA), or the like. The terminal device 8 is provided with a sensor for measuring a lifelog of a user, and transmits, to the state prediction device 2A, a measurement signal output from the sensor, an input signal based on a user input, or the like.

The state prediction device 2A has a hardware configuration illustrated in FIG. 2A, and functional block configurations illustrated in FIGS. 4 and 7. Then, after executing processing of training a state prediction model, the state prediction device 2A predicts a state using the state prediction model and recommends an action amount using a recommendation model. In this case, the state prediction device 2A (specifically, UI control unit 29 in FIG. 7) transmits, to the terminal device 8 via the network 7, output signals related to a state prediction result and a recommended action amount in response to a request from the terminal device 8. In this case, the terminal device 8 functions as the output device 5 in the first example embodiment.

As described above, the state prediction system 100A according to the second example embodiment is capable of training a state prediction model and predicting a state using the trained state prediction model, and is capable of suitably presenting a result of the state prediction and the like to the user of the terminal device 8. In the second example embodiment, a device different from the state prediction device 2A may execute the processing of training the recommendation model.

### Third Example Embodiment

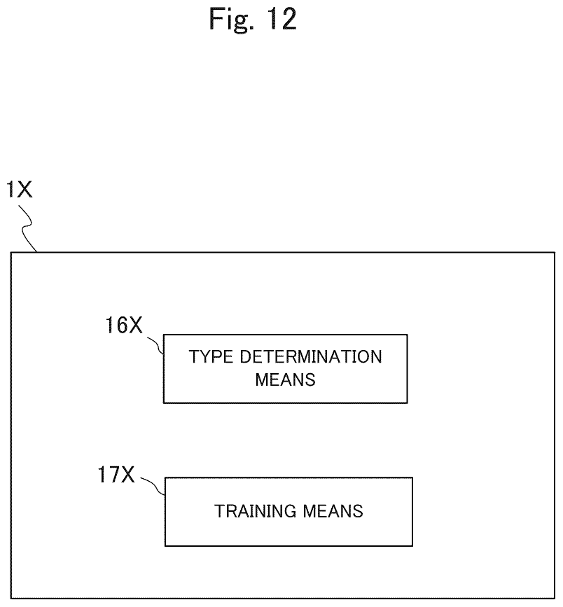

FIG. 12 illustrates a block diagram of a training device 1X. The training device 1X mainly includes, a type determination means 16X and a training means 17X. The training device 1X may be configured by plural devices.

The type determination means 16X is configured to determine a type of each of subjects related to an action based on a recommended action, which is an amount of the action recommended to each of the subjects, a type of the action, or a combination of the amount of the action and the type of the action, and an actual action, which is an actual amount of the action of each of the subjects, a type of the action, or a combination of the actual amount of the action and the type of the action. Examples of the type determination means 16X include the subject type determination unit 16 of the training device 1 according to the first example embodiment and the subject type determination unit 16 of the state prediction device 2A according to the second example embodiment.

The training means 17X is configured to train a state prediction model, for each type related to the action, based on a lifelog of each of the subjects classified by the type, wherein the state prediction model is obtained by performing machine learning of a relationship between a lifelog and a predicted index value of a state to be predicted of a person for which the lifelog has been measured. Examples of the training means 17X include the type-specific training unit 17 of the training device 1 according to the first example embodiment and the type-specific training unit 17 of the state prediction device 2A according to the second example embodiment.

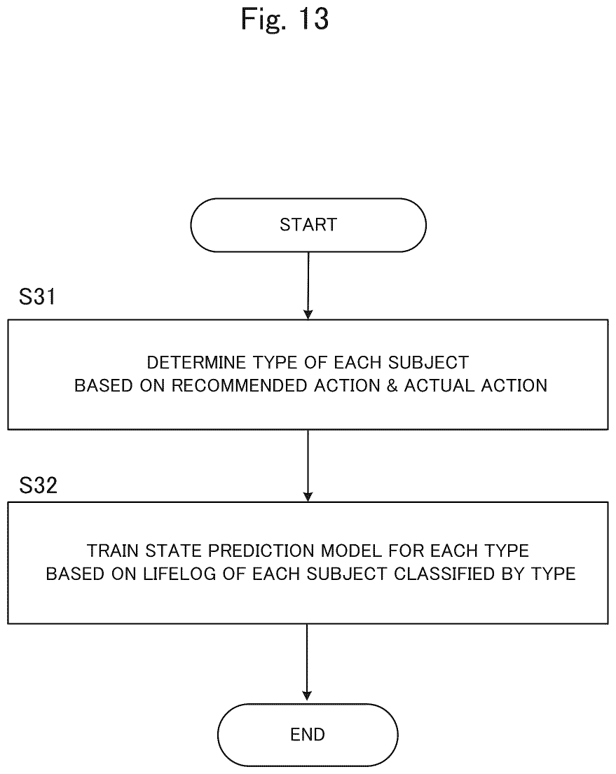

FIG. 13 is an example of a flowchart executed by the training device 1X. The type determination means 16X determines a type of each of subjects related to an action based on a recommended action, which is an amount of the action recommended to each of the subjects, a type of the action, or a combination of the amount of the action and the type of the action, and an actual action, which is an actual amount of the action of each of the subjects, a type of the action, or a combination of the actual amount of the action and the type of the action (step S31). Then, the training means 17X trains a state prediction model, for each type related to the action, based on a lifelog of each of the subjects classified by the type (step S32). The above-mentioned state prediction model is obtained by performing machine learning of a relationship between a lifelog and a predicted index value of a state to be predicted of a person for which the lifelog has been measured.

According to the third example embodiment, the training device 1X can train the state prediction model for accurately predicting the state of the subject by using the lifelog.

### Fourth Example Embodiment

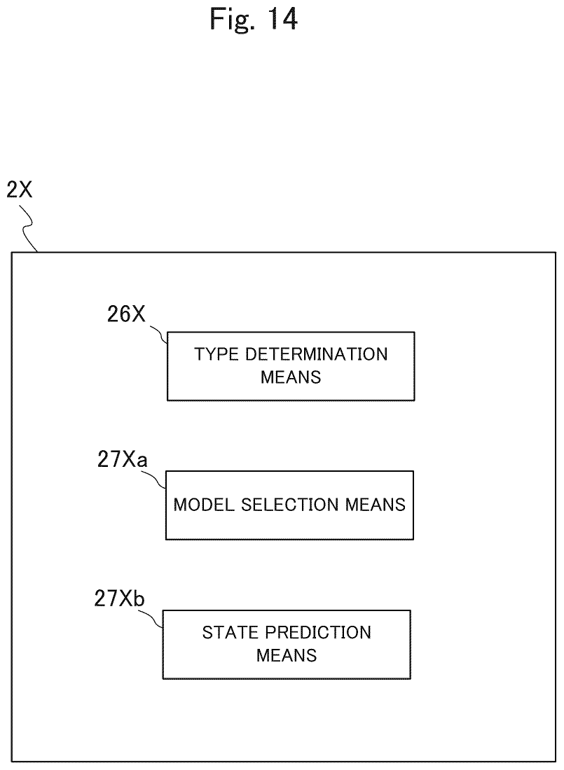

FIG. 14 is a block diagram of a state prediction device 2X. The state prediction device 2X mainly includes a type determination means 26X, a model selection means 27Xa, and a state prediction means 27Xb. The state prediction device 2Y may be configured by plural devices.

The type determination means 26X is configured to determine a type of a subject related to an action, based on a recommended action, which is an amount of the action recommended to the subject, a type of the action, or a combination of the amount of the action and the type of the action, and an actual action, which is an actual amount of the action of the subject, a type of the action, or a combination of the actual amount of the action and the type of the action.

Examples of the type determination means 26X include the subject type determination unit 26 of the state prediction device 2 according to the first example embodiment and the subject type determination unit of the state prediction device 2A according to the second example embodiment.

The model selection means 27Xa is configured to select a state prediction model to be used in state prediction of the subject from state prediction models trained for each type related to the action based on the determined type, wherein the state prediction model is obtained by performing machine learning of a relationship between a lifelog and a predicted index value of a state to be predicted of a person for which the lifelog has been measured.

The state prediction means 27Xb is configured to predict a state of the subject based on the selected state prediction model and a lifelog of the subject. The model selection means 27Xa and the state prediction means 27Xb may be the state prediction unit 27 of the state prediction device 2 according to the first example embodiment or the state prediction unit 27 of the state prediction device 2A according to the second example embodiment.

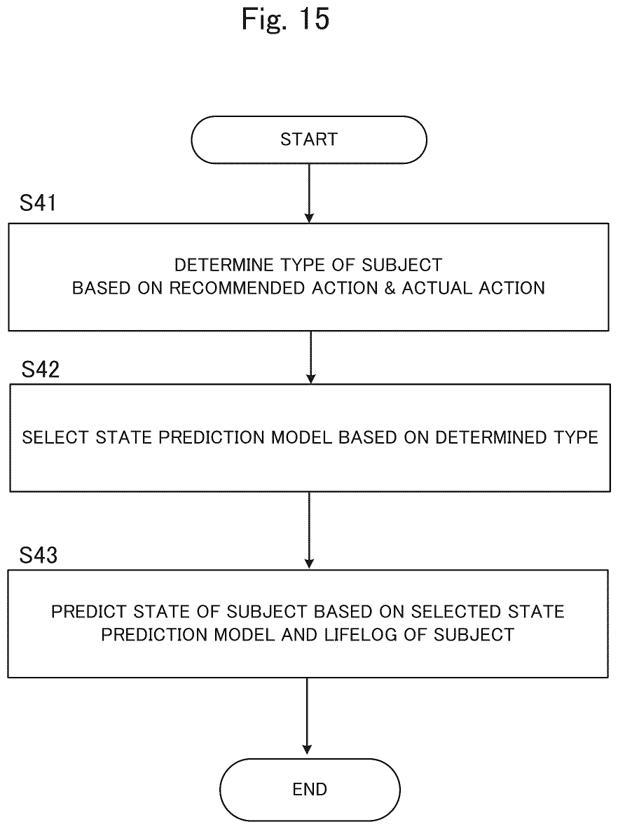

FIG. 15 is an example of a flowchart executed by the state prediction device 2X. The type determination means 26X determines a type of a subject related to an action, based on a recommended action, which is an amount of the action recommended to the subject, a type of the action, or a combination of the amount of the action and the type of the action, and an actual action, which is an actual amount of the action of the subject, a type of the action, or a combination of the actual amount of the action and the type of the action (step S41). Then, the model selection means 27Xa selects a state prediction model to be used in state prediction of the subject from state prediction models trained for each type related to the action based on the determined type, wherein the state prediction model is obtained by performing machine learning of a relationship between a lifelog and a predicted index value of a state to be predicted of a person for which the lifelog has been measured (step S42). The state prediction means 27Xb predicts a state of the subject based on the selected state prediction model and a lifelog of the subject (step S43).

The state prediction device 2X according to the fourth example embodiment suitably selects a state prediction model in accordance with the type of the subject determined based on whether or not a recommended action has been taken, which leads to accurate prediction of the state of the subject.

In the example embodiments described above, the program is stored by any type of a non-transitory computer-readable medium (non-transitory computer readable medium) and can be supplied to a control unit or the like that is a computer. The non-transitory computer-readable medium include any type of a tangible storage medium. Examples of the non-transitory computer readable medium include a magnetic storage medium (e.g., a flexible disk, a magnetic tape, a hard disk drive), a magnetic-optical storage medium (e.g., a magnetic optical disk), CD-ROM (Read Only Memory), CD-R, CD-R/W, a solid-state memory (e.g., a mask ROM, a PROM (Programmable ROM), an EPROM (Erasable PROM), a flash ROM, a RAM (Random Access Memory)). The program may also be provided to the computer by any type of a transitory computer readable medium. Examples of the transitory computer readable medium include an electrical signal, an optical signal, and an electromagnetic wave. The transitory computer readable medium can provide the program to the computer through a wired channel such as wires and optical fibers or a wireless channel.

In addition, some or all of the above-described example embodiments (including modifications, the same shall apply hereinafter) may also be described as follows, but are not limited to the following. Furthermore, within the range defined by the above-described example embodiments, regardless of the device, method, and storage medium described in the following Supplementary Notes, some or all of the configurations described in the following Supplementary Notes may be applied to any hardware, software, system and recording means (including the storage medium) for recording a software.

### [Supplementary Note 1]

A training device comprising:
a type determination means for determining a type of each of subjects related to an action, based on
   a recommended action, which is an amount of the action recommended to each of the subjects, a type of the action, or a combination of the amount of the action and the type of the action, and
   an actual action, which is an actual amount of the action of each of the subjects, a type of the action, or a combination of the actual amount of the action and the type of the action; and
a training means for training a state prediction model, for each type related to the action, based on a lifelog of each of the subjects classified by the type, wherein
the state prediction model is obtained by performing machine learning of a relationship between a lifelog and a predicted index value of a state to be predicted of a person for which the lifelog has been measured.

### [Supplementary Note 2]

The training device according to Supplementary Note 1, wherein the type determination means determines whether each of the subjects has complied with the recommended action based on the recommended action and the actual action, and determines the type based on a result of the determination as to whether each of the subjects has complied with the recommended action.

### [Supplementary Note 3]

The training device according to Supplementary Note 2, wherein the type determination means further determines at least one of a gender or an age of each of the subjects, and determines the type based on the result of the determination as to whether each of the subjects has complied with the recommended action and a result of the determination as to at least one of the gender or the age.

### [Supplementary Note 4]

The training device according to Supplementary Note 1, wherein the training means trains a plurality of the state prediction models having a different length from a prediction time point at which the state is predicted using the state prediction model to a predicted time point at which the state is predicted.

### [Supplementary Note 5]

A state prediction device comprising:
a type determination means for determining a type of a subject related to an action, based on
   a recommended action, which is an amount of the action recommended to the subject, a type of the action, or a combination of the amount of the action and the type of the action, and
   an actual action, which is an actual amount of the action of the subject, a type of the action, or a combination of the actual amount of the action and the type of the action;
a model selection means for selecting a state prediction model to be used in state prediction of the subject from state prediction models trained for each type related to the action based on the determined type; and
a state prediction means for predicting a state of the subject based on the selected state prediction model and a lifelog of the subject, wherein
the state prediction model is obtained by performing machine learning of a relationship between a lifelog and a predicted index value of a state to be predicted of a person for which the lifelog has been measured.

### [Supplementary Note 6]

The state prediction device according to Supplementary Note 5, further comprising a recommendation means for calculating the recommended action to be newly recommended to the subject based on the lifelog of the subject.

### [Supplementary Note 7]

The state prediction device according to Supplementary Note 6, further comprising an output control means for outputting, using an output device, a result of the prediction of the state and the recommended action calculated by the recommendation means.

### [Supplementary Note 8]

The state prediction device according to Supplementary Note 5, further comprising a lifelog acquisition means for obtaining the lifelog of the subject in a predetermined period immediately before a prediction time point, wherein
the state prediction means generates a lifelog from the prediction time point to a predicted time point at which the state is predicted based on the lifelog in the predetermined period, and predicts the state of the subject based on the lifelog in the predetermined period, the lifelog from the prediction time point to the predicted time point, and the state prediction model.

### [Supplementary Note 9]

A training method executed by a computer, comprising:
determining a type of each of subjects related to an action, based on
   a recommended action, which is an amount of the action recommended to each of the subjects, a type of the action, or a combination of the amount of the action and the type of the action, and
   an actual action, which is an actual amount of the action of each of the subjects, a type of the action, or a combination of the actual amount of the action and the type of the action; and
training a state prediction model, for each type related to the action, based on a lifelog of each of the subjects classified by the type, wherein
the state prediction model is obtained by performing machine learning of a relationship between a lifelog and a predicted index value of a state to be predicted of a person for which the lifelog has been measured.

### [Supplementary Note 10]

A program executed by a computer, the program causing the computer to:
determine a type of each of subjects related to an action, based on
   a recommended action, which is an amount of the action recommended to each of the subjects, a type of the action, or a combination of the amount of the action and the type of the action, and
   an actual action, which is an actual amount of the action of each of the subjects, a type of the action, or a combination of the actual amount of the action and the type of the action; and
train a state prediction model, for each type related to the action, based on a lifelog of each of the subjects classified by the type, wherein
the state prediction model is obtained by performing machine learning of a relationship between a lifelog and a predicted index value of a state to be predicted of a person for which the lifelog has been measured.

### [Supplementary Note 11]

A state prediction method executed by a computer, comprising:
determining a type of a subject related to an action, based on
   a recommended action, which is an amount of the action recommended to the subject, a type of the action, or a combination of the amount of the action and the type of the action, and
   an actual action, which is an actual amount of the action of the subject, a type of the action, or a combination of the actual amount of the action and the type of the action;
selecting a state prediction model to be used in state prediction of the subject from state prediction models trained for each type related to the action based on the determined type; and
predicting a state of the subject based on the selected state prediction model and a lifelog of the subject, wherein
the state prediction model is obtained by performing machine learning of a relationship between a lifelog and a predicted index value of a state to be predicted of a person for which the lifelog has been measured.

### [Supplementary Note 12]

A program executed by a computer, the program causing the computer to:
determine a type of a subject related to an action, based on
   a recommended action, which is an amount of the action recommended to the subject, a type of the action, or a combination of the amount of the action and the type of the action, and
   an actual action, which is an actual amount of the action of the subject, a type of the action, or a combination of the actual amount of the action and the type of the action;
select a state prediction model to be used in state prediction of the subject from state prediction models trained for each type related to the action based on the determined type; and
predict a state of the subject based on the selected state prediction model and a lifelog of the subject, wherein
the state prediction model is obtained by performing machine learning of a relationship between a lifelog and a predicted index value of a state to be predicted of a person for which the lifelog has been measured.

### [Supplementary Note 13]

A storage medium storing a program according to Supplementary Note 10 or 12.

While the invention has been particularly shown and described with reference to example embodiments thereof, the invention is not limited to these example embodiments. It will be understood by those of ordinary skilled in the art that various changes in form and details may be made therein without departing from the spirit and scope of the present invention as defined by the claims. In other words, it is needless to say that the present invention includes various modifications that could be made by a person skilled in the art according to the entire disclosure including the scope of the claims, and the technical philosophy. Each example embodiment can be appropriately combined with other example embodiments. All Patent and Non-Patent Literatures mentioned in this specification are incorporated by reference in its entirety.

### DESCRIPTION OF REFERENCE NUMERALS

1, 1X Training device
2, 2A, 2X State prediction device
3 Storage device
4 Input device
5 Output device
8 Terminal device
100, 100A State prediction system

1. A training device comprising:
at least one memory configured to store instructions; and
at least one processor configured to execute the instructions to:
determine a type of each of subjects related to an action, based on
a recommended action, which is an amount of the action recommended to each of the subjects, a type of the action, or a combination of the amount of the action and the type of the action, and
an actual action, which is an actual amount of the action of each of the subjects, a type of the action, or a combination of the actual amount of the action and the type of the action; and
train a state prediction model, for each type related to the action, based on a lifelog of each of the subjects classified by the type, wherein
the state prediction model is obtained by performing machine learning of a relationship between a lifelog and a predicted index value of a state to be predicted of a person for which the lifelog has been measured.

2. The training device according to claim 1, wherein the at least one processor is configured to execute the instructions to determine whether each of the subjects has complied with the recommended action based on the recommended action and the actual action, and determines the type based on a result of the determination as to whether each of the subjects has complied with the recommended action.

3. The training device according to claim 2, wherein the at least one processor is configured to execute the instructions to further determine at least one of a gender or an age of each of the subjects, and determine the type based on the result of the determination as to whether each of the subjects has complied with the recommended action and a result of the determination as to at least one of the gender or the age.

4. The training device according to claim 1, wherein the at least one processor is configured to execute the instructions to train a plurality of the state prediction models having a different length from a prediction time point at which the state is predicted using the state prediction model to a predicted time point at which the state is predicted.

5. A state prediction device comprising:
at least one memory configured to store instructions; and
at least one processor configured to execute the instructions to:
determine a type of a subject related to an action, based on
a recommended action, which is an amount of the action recommended to the subject, a type of the action, or a combination of the amount of the action and the type of the action, and
an actual action, which is an actual amount of the action of the subject, a type of the action, or a combination of the actual amount of the action and the type of the action;
select a state prediction model to be used in state prediction of the subject from state prediction models trained for each type related to the action based on the determined type; and
predict a state of the subject based on the selected state prediction model and a lifelog of the subject, wherein
the state prediction model is obtained by performing machine learning of a relationship between a lifelog and a predicted index value of a state to be predicted of a person for which the lifelog has been measured.

6. The state prediction device according to claim 5, wherein the at least one processor is configured to execute the instructions to further calculate the recommended action to be newly recommended to the subject based on the lifelog of the subject.

7. The state prediction device according to claim 6, wherein the at least one processor is configured to execute the instructions to further output, using an output device, a result of the prediction of the state and the recommended action.

8. The state prediction device according to claim 5, the at least one processor is configured to execute the instructions to further obtain the lifelog of the subject in a predetermined period immediately before a prediction time point, wherein
the at least one processor is configured to execute the instructions to generate a lifelog from the prediction time point to a predicted time point at which the state is predicted based on the lifelog in the predetermined period, and predicts the state of the subject based on the lifelog in the predetermined period, the lifelog from the prediction time point to the predicted time point, and the state prediction model.

9. A training method executed by a computer, comprising:
determining a type of each of subjects related to an action, based on
a recommended action, which is an amount of the action recommended to each of the subjects, a type of the action, or a combination of the amount of the action and the type of the action, and
an actual action, which is an actual amount of the action of each of the subjects, a type of the action, or a combination of the actual amount of the action and the type of the action; and
training a state prediction model, for each type related to the action, based on a lifelog of each of the subjects classified by the type, wherein
the state prediction model is obtained by performing machine learning of a relationship between a lifelog and a predicted index value of a state to be predicted of a person for which the lifelog has been measured.
